(19) United States
(12) Patent Application Publication
(10) Pub. No.: US 2008/0318310 A1
(43) Pub. Date: Dec. 25, 2008

(54) Method and Apparatus for Automatically Isolating Microbial Species

(75) Inventors: Claude Dufresne, East Brunswick, NJ (US); Gary S. Kath, Scotch Plains, NJ (US); Gregory W. King, Carteret, NJ (US); Prakash S. Masurekar, Warren, NJ (US); Salvatore J. Siciliano, East Brunswick, NJ (US); Janet M. Sigmund, Cranford, NJ (US); Neal R. Simpson, Frenchtown, NJ (US); Stephen Skwish, East Brunswick, NJ (US)

Correspondence Address: MERCK AND CO., INC, P O BOX 2000, RAHWAY, NJ, 07065-0907, US

(73) Assignee: MERCK & CO., INC., Rahway, NJ (US)

(21) Appl. No.: 11/664,486

(22) PCT Filed: Oct. 7, 2005
(86) PCT No.: PCT/US05/36596

§ 371 Date: May 5, 2008

Related U.S. Application Data
(60) Provisional application No. 60/618,370, filed on Oct. 13, 2004.

(51) Int. Cl. C12M 1/26

(52) U.S. Cl. 435/309.1

(57) ABSTRACT

This invention relates to an apparatus for streaking a sample on a media-coated (agar) plate in a predefined pattern utilizing a plate carousel system to house and retrieve a plate to a fixed location, a streaking mechanism having a floating stylus having a tip capable of gliding over the agar surface and depositing a sample onto the surface of the agar, a sample dispensing system and an optional wash station.

### FIELD OF THE INVENTION

The present invention relates to an apparatus and method for automatically transferring bacterial specimens from specimen containers to the surface of culture medium plates in programmable patterns and concentrations (streaking) to produce isolated bacterial colonies. The present invention provides for the precise deposition of an inoculant at a specific location and concentration on the surface of a culturing medium.

### BACKGROUND OF THE INVENTION

The isolation of a sample of a bacterial specimen consists of the inoculation of the sample on a plate containing culture medium and requires that the specimen sample be spread or “streaked” over the plates containing culture medium in a prescribed pattern that is correlated to the specific specimen. The patterns provide an increasing dilution of the sample and are affected by a streaking tool. The streaked plate is then incubated to promote bacterial growth into isolated microbial colonies. The microbial isolated colonies can then be examined or subjected to further testing for infectious diseases or identification.

Traditionally, the streaking operation is completed manually utilizing a variety of sterilized stylus, for example, wire loops, glass pipettes, or cotton swabs. This process is often tedious and very time consuming. Additionally, the effectiveness of the streaking operation is subject to the particular techniques and skills of the technicians performing the procedure. In a manual operation it would be difficult to maintain consistency between the techniques used by different technicians or even between different samples prepared by the same technician at different times.

Attempts have been made to automate the streaking process. A commercial spiral plater, Spiral Biotech, Inc. Norwood, Mass.; AUTOPLATE 4000®, has been developed to deposit precisely controlled amounts of liquid sample in a spiral pattern onto the surface of a rotating agar plate. The spiral plater is often used to prepare plates for aerobic plate counting (APC) procedures for determining the level of microorganism in a food product. The spiral plater accepts only 100 and 150 mm circular Petri plates. There are only two plate spreading speeds, normal (2.5 rps) and slow (1.5 rps) and the system has only one syringe choice with a capacity of 250 μL. These limitations restrict adjustment of the concentration range of the sample that is deposited on the agar. Further more, an automated plate feeder is not available with this spiral plater system. Additionally, the spiral plater system can not process other shaped plates, for example, rectangular plates.

It would be beneficial to develop a method and apparatus that is capable of processing batches of plates, such as for example, SBS standard rectangular microplates, unattended and having the following attributes: accommodates a plurality of microplates, incorporates a stylus capable of moving across the agar medium in any direction, and distributes a varied array of sample concentrations onto the agar in a variety of streaking patterns.

### SUMMARY OF THE INVENTION

The present invention relates to an apparatus for an unmanned, unattended method to streak varying concentrations of natural product samples onto microtier size agar plates. The apparatus, a semi-automated agar plate streaker, takes up liquid samples and streaks the liquid samples over nutrient media in a predefined path at a varying volume per unit streak length. The streak concentration (volume per unit streak length) can vary over 3 orders of magnitude over a given plate.

The combination of different starting liquid sample concentrations and the exponential streak concentration process aids in the separation of colonies for visual inspection of effectiveness against infectious diseases. Isolated colonies of interest can be manually removed from the streaked plates for regrowth.

The apparatus of the present invention comprises a) a plate carousel system, capable of storing a plurality of plates and retrieving a given plate to a fixed location, b) a streaking mechanism directly attached to the plate carousel system, c) a sample dispensing system, and d) a wash station.

The streaking mechanism is attached directly to the deck of a plate carousel system such as, for example, a TECAN SLT L® carousel. The agar plates are un-lidded prior to being placed in the carousel. A plate is picked from the carousel and placed onto a picking platform, the plate remains on the picking platform during streaking process.

The carousel system design incorporates a plurality of removable multi-compartmental cassettes that houses a plate in each compartment. The cassettes are connected to a rotary platform that presents the plates to an integral plate picker mechanism. The plate picker retrieves a selected plate from a stack and presents it to a fixed location. The random access time is variable and allows for flexibility in the sequence of the plate selection.

The streaking mechanism comprises a coupled multi-axis, XYZ-Theta, motion system with a floating stylus. To achieve uniform streaking of the sample and to glide over agar surface variations, an engineered stylus tip is mounted to a bearing assembly. Samples are supplied to the stylus by way of a pump that moves the liquid sample from the sample reservoir to the stylus via tubing. From the stylus, the sample is delivered to the agar via a predefined pattern and flowrate. The patterns provide an increasing dilution of the sample and are affected by the stylus.

A server, such as a personal computer, is used to control the hardware and fluid components of the system via multiple RS232 control ports and affords the operator great flexibility in establishing operating parameters such as the capability to streak various sample concentrations, select different sample volumes, and select varying streaking speeds.

### BRIEF DESCRIPTION OF THE DRAWINGS

The above and other objects, features, advantages and technical significance of the present invention will be better understood by reading the following detailed description of preferred embodiments of the invention, when considered in connection with the accompanying drawings, in which:

FIG. 1 depicts a general overview of the plate streaking system components.

FIG. 2 represents a side perspective of the stylus.

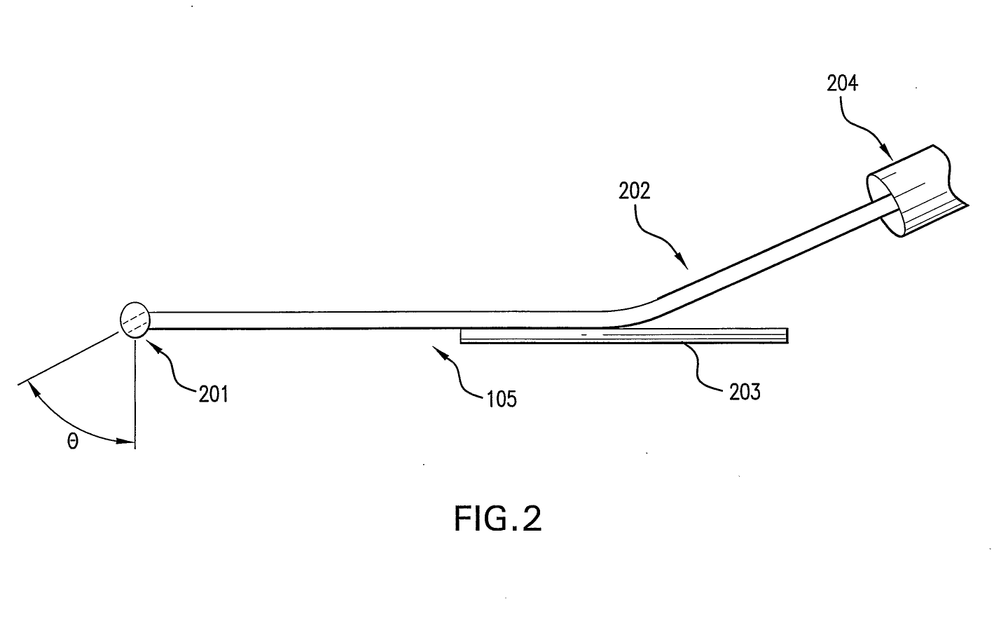

FIG. 3 represents a frontal view of the streaking mechanism.

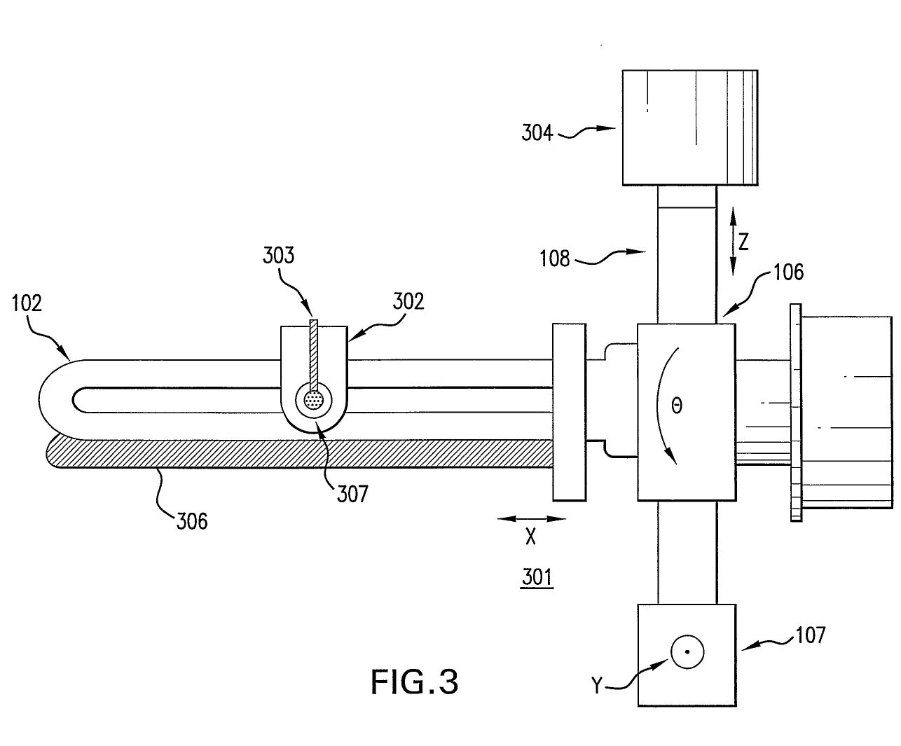

FIG. 4 represents a top, planar view of the streaking mechanism.

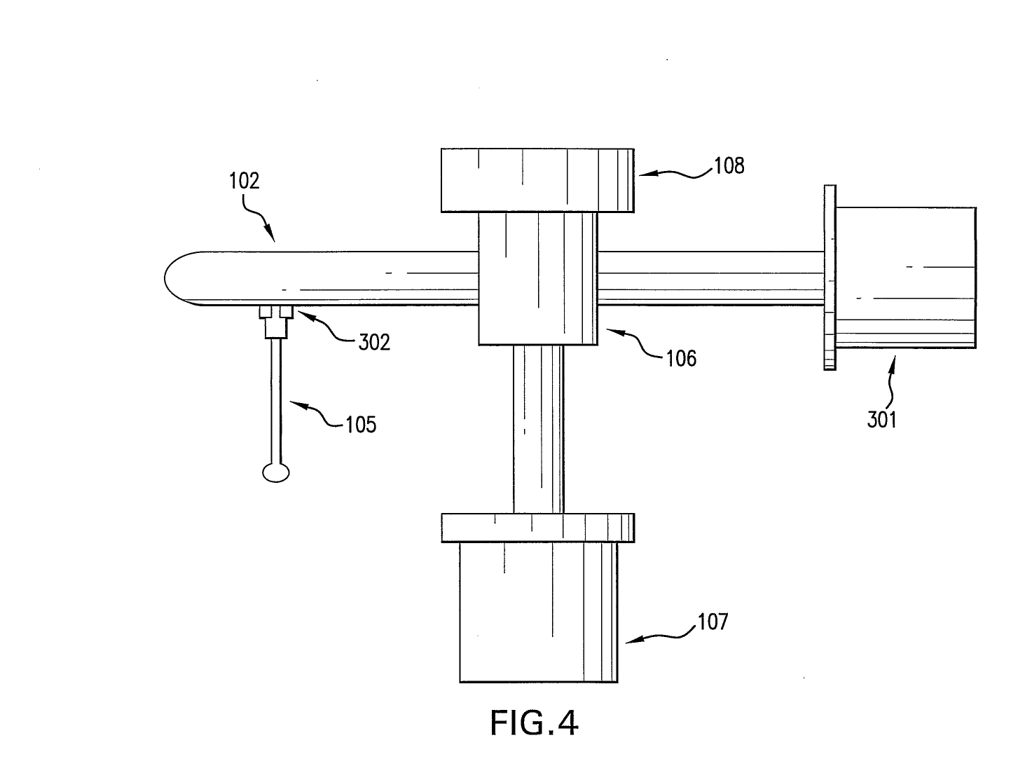

FIG. 5 depicts a general overview of a plate streaking system containing a wash station and a sample station.

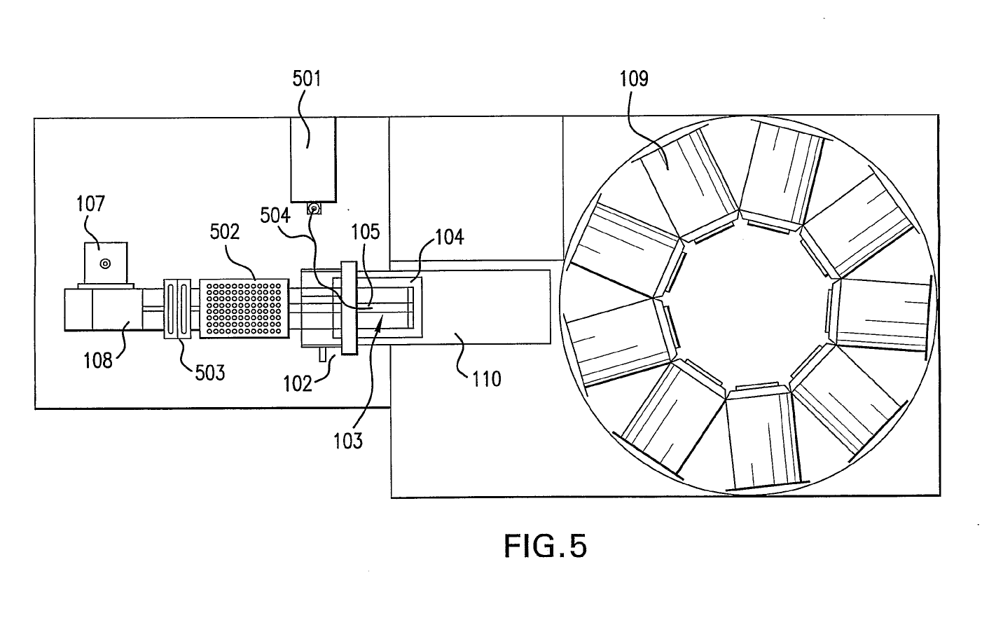

### DETAILED DESCRIPTION OF THE INVENTION

The present invention is more particularly described in the following examples that are intended as illustrative only since numerous modifications and variations therein will be apparent to those skilled in the art. As used in the specification and in the claims, “a”, “an,” and “the” can mean one or more, depending upon the context in which it is used. Embodiments of the invention are described with reference to the Figures, in which like numbers indicate like parts throughout the figures.

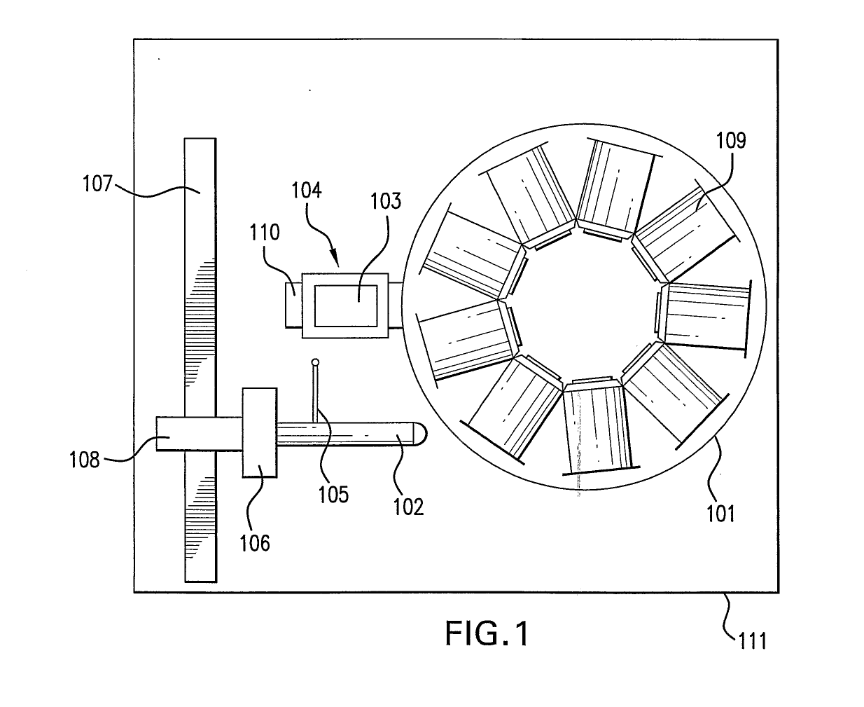

FIG. 1 gives an overview of the semi-automated plate streaker system of the present invention. A carousel hotel storage system (101) stores and retrieves a plate (103) onto a picking platform (104) and to a fixed location for further processing. The carousel has a plurality of removable multi-compartmental plate hotels (109). Each plate hotel (109) has the capacity to house multiple plates. Many variants of the carousel design are possible, but the carousel system should have the capability to: choose one specific plate from any one of the various hotels, retrieve the plate, and delivery the plate to a fixed location. The plate streaker system is mounted on a stable base (111). The picking platform (104) is actuated via the picking platform mechanism (110).

In one embodiment of the invention commercially available plate storage and delivery carousel, TECAN U.S. SLT L®, Carousel Model 61-506, is utilized. The TECAN unit has the capability to hold up to ninety-nine rectangular-shaped (SBS standard) plates. The carousel has nine vertical plate hotels attached to a rotary axis. Each plate hotel holds eleven plates. The carousel can be controlled via a RS-232 interface using ASCII commands that allow rotation of the carousel and retrieval of a plate from any hotel to the work zone (fixed location). The carousel control unit can easily be integrated with the other elements of the plate streaker system via a computer system. Additionally, the carousel unit can be mounted on a stable base.

Once a plate (103) is in the fixed location position, the streaking mechanism is employed. The streaking mechanism provides a coupled multi-axis, XYZ-Theta, motion system with a floating stylus (105). The floating stylus comprises a stylus tip mounted to a bearing assembly. Use of the XYZ-Theta configuration allows for very rapid stylus acceleration/deceleration, high velocity stylus speeds and the capability of synchronization with a sample delivery pump such as a syringe pump. The XYZ-Theta motion system makes it possible to streak in any interpolation. These properties make it possible for the stylus to streak in a rectangular format. That is, streak in a series of parallel lines wherein each line contains a different concentration of deposited sample. Additionally, the high speed drives coupled with the variable speed syringe pumping system make it possible to obtain a wide range of sample dilutions.

The rotary (theta) axis (106) is integral to maintaining multiple processes as well as for organizing motion within small footprint area. Functions of the rotary (theta) axis (106) include: rotating vertically to a hard stop locking the floating stylus (105) to allow vertical access into sample tubes or a wash station; rotating horizontally to home the axis, and rotating to an angle to deliver sample and to allow the floating stylus (105) to contact the media deposited on the surface of the plate (103). An example of a rotary (theta) axis which can be employed in the system is the Model B5990TS manufactured by Vermex Inc., Bloomfield N.Y.

For the X axis (301), a lead screw linear slide is employed to maximize positioning accuracy and operating life. The linear motion slides are driven by servo motors. The travel speed of the slide is in part determined by the diameter and pitch of the screw selected. For example, for high speed travel in the X direction, a screw having a diameter ranging from about 0.125 inches to about 0.75 inches and a pitch ranging from about 0.03125 inches to about 1.5 inches can be utilized. In one embodiment of the invention, a screw having a diameter of about 0.25 inches and a pitch of about 1 inch is employed. For smooth, quiet, long-life operation, the lead screw nut is made from material such as for example, acetal. The acetal lead screw nut also provides the advantage that it is self-lubricating.

The linerar screw slide is housed inside a cylindrical tube (102) having two slots machined along its length. Attached to the lead screw nut is a custom acetal bushing with minimal clearance to the inner diameter of the stainless steel cylindrical tube to prevent the lead screw from whipping at high traverse speeds. This bushing has a cross pin press fitted into its circumference which is assembled via a slip fit though the slot of the stainless steel cylindrical tube. The cross pin keeps the bushing from turning in the stainless steel cylindrical tube and helps advancement along the drive screw. Additionally, the cross pin acts as a vane when homing.

The Y and Z axis ((107) and (108), respectively) are also equipped with ball screw technology for precision. Suitable commercially available examples of ball screw slides which can be employed in the present invention include those offered by NSK Corporation, Bloomingdale Ill., Models MCM05015H05K and MCM05040H10K. As is the case with the X axis slide, both the Y and Z axis slides can be equipped with variously pitched lead screws depending on traverse speed desired. For example, in one embodiment, the Y axis slide is equipped with a 1 cm pitch lead screw and the Z axis slide is equipped with a 0.5 cm pitch screw. Here, the Y axis slide allows the stylus to glide across the surface of the media deposited on the plate. The Z axis slide (108) allows the stylus to be lowered and lifted in the vertical direction, thus making access to sample containers, wash stations, and media surface possible.

Attached to the X axis lead screw nut is a floating tip mechanism. The floating tip mechanism provides the streaker system with the ability to track over and compensate for surface or thickness variations of the deposited media. Construction of the floating tip mechanism comprises press fitting a bearing onto the end of the X axis lead screw nut. An aluminum bushing is press fitted onto the outer diameter of the bearing. The aluminum bushing is crossed drilled on the circumference. A fabricated clamping device collet (302) attaches to the aluminum bearing to allow easy removal of a stylus (105). The stylus (105) is mounted to the collet thus completing the floating tip mechanism.

The stylus (105) comprises a stainless steel ball (201) having a cross drilled hole and is laser welded on the one end of syringe stock (202). A hardened drill blank (203) is welded onto the opposite end for mounting ease. The laser welded ball is the applicator end of the stylus (105). The gauge of the syringe stock and the size of the stainless steel ball are variable and are based on the viscosity and particulate content of the sample to be streaked. For routine operations, sixteen gauge syringe stock and a 0.125 inch diameter stainless steel ball is used. A tube fitting (204) is then attached to the syringe stock open end with a predetermined dead volume loop of tubing. The tubing is then attached to a programmable syringe pump.

The ball (201) on the applicator end of the stylus (105) is highly polished and has the ability to float harmlessly over the agar in any programmable interpolation. The ball tip permits multidirectional movement of the stylus over the agar surface. The cross drilled hole is maintained perpendicular to the agar surface. This aids in the uniform delivery of any particulate matter which may be contained in the liquid sample.

The samples to be streaked are aspirated and dispensed via a programmable syringe pump which connects to the stylus end (204) via tubing. TEFLON tubing is one example of acceptable tubing material which can be utilized. Optionally, a syringe (for example, having 250 μL or 500 μL capacity) can be inserted in the tubing located between the stylus (105) and the syringe pump (501) to act as a storage reservoir. This conveniently allows a variable amount of sample to be streaked before the refilling of the syringe is necessary.

To wash the stylus (105) between samples, a wash station (503) can be incorporated into the streaking system of the present invention. In one embodiment of the invention, the wash station (503) comprises a wash zone having high flow rate piston pumps to rapidly purge the wash area and provide a clean fountain wash for the exterior of the stylus. Wash solution is pumped up through wells to form a fountain effect. Additionally, solenoid valves can be utilized to direct the high flow rate pumping to wash the interior of the stylus. Around the perimeter of the wash wells, a drain zone can be incorporated to contain the fountain overflow. Waste from the drain is gravity fed to a waste container. The waste container is equipped with a liquid level sensor which alarms when the container is full.

Optionally, a stylus drying zone is provided by drawing high velocity air past the stylus (105) into a vacuum trap to remove the last drop of liquid that might be left behind prior to the retrieval of the next sample.

In one embodiment of the invention, a multi-port wash/dry/waste station is employed.

A server, such as a personal computer, is used to control the hardware and fluidics via two RS232 control ports. One RS-232 port connects to the TECAN carrousel and the other to the addressable bank of multiple servo motors. An interface software package is used on the front end to enable the user to accommodate their particular protocol by inputting a variety of parameters such as flow rates, streaking pattern, plate selection, etc. Incorporated into the system is an algorithm giving the ability to streak various sample concentrations, sample volume, and streaking speeds. For example, the system can be instructed to dilute sample 100 to 1 or to dispense a constant concentration across the entire plate. The system also has the capability to setup multiple sequences per run. The software system allows numerous streaking protocols to be stored and be conveniently initialized at a later time without having to reprogram the individual steps. In this manner, multiple plates with various streaking sequences can be run automatically.

Various pre-programmed operational modes such as a specific priming sequence of the system fluid lines to a waste basin, a system start mode, and system shutdown mode, a stylus wash mode, or a system debug mode used for testing of the different hardware components can be stored and later called up by the operator. Each pre-programmed mode provides instructions to the various hardware components regarding the precise sequence of coordinated equipment manipulations necessary to perform a particular task. The system can optionally be provided with an emergency stop and reset sequence which respectively, safely powers down and restarts the mechanical equipment in case of emergency.

The streaker system operates automatically and unattended preparing one or more plates per run. The system has the ability to streak the plates in any programmed interpolation. The operator has the flexibility to program the streaking pattern, the number of streaks, and the amount and concentration of sample to be deposited.

Typical initial startup procedures are described below. The system is energized by reseting the emergency stop if it was previously activated and then booting up the power supply and server. When the energizing of the system is complete, the rotary (theta) axis (106) homes itself horizontally, the XYZ axis are each homed as well. Through an interactive interface, an operator will input answers to a series of prompts generated by a computer program designed to determine various operating parameters, such as for example, the number of plates to streak, determination of sample selection, determination of dispensing sequence, determination of deposited sample concentration, determination of stylus speed, etc. The plates (103) are then manually loaded into the carousel storage system (101). The rotary (theta) axis (106) can then be rotated to an angle ranging from 0 to 90 degrees against a physical stop bar (306). For example, the rotary axis (106) can be rotated 90 degrees against a physical stop bar (306), thus locking the floating stylus mechanism in the vertical plain. This position affords precision access to the wash locations (503) and sample containers (502). The system can be primed and a stylus wash sequence can be performed if desired. Next a sample is aspirated into the dead volume tubing loop (504) (and optional reservoir syringe if desired) using the programmable syringe pump (501) in preparation to streak a sample. The rotary axis (106) rotates to a desired angle, typically 45 degrees, with the Z axis (108) gently lowering the stylus tip (201) onto the media surface. The rotary axis (106) then rotates slightly to enable the floating mechanism to function via gravity.

A predefined streaking protocol is then executed. In one embodiment of the invention, the streaking pattern begins with the stylus tip (201) touching down on the media covered surface of the plate (103) in the upper left quadrant of a plate. The stylus (105) then streaks from right to left, indexing each pass in the Y negative direction until the entire surface is covered. The number of streaks per plate and the sample dilution is predefined via operator input.

Between the return of the streaked plate and the delivery of a new, unstreaked plate, an optional wash sequence can be performed. To accomplish this, the floating stylus mechanism is maneuvered over the wash station and the stylus (105) is lowered into a fountain wash station (503). A vacuum source is located at one area of the wash station (503). The vacuum is used to rid the stylus tip of any residual liquid that may remain. Once the wash/dry sequence is complete, the plate (103) is returned to its original slot in the carousel storage system (101). Another plate (103) can then be selected from the hotel (109) and the whole streaking process can begin again until the predefined program is complete.

Upon completion of the run, the carousel is manually unloaded and the plates are covered and returned to incubation. The shutdown sequence can then be initiated by flushing and cleaning the streaking system.

What is claimed is:

1. An apparatus for streaking a sample on a plate comprising a) a plate carousel system, capable of storing a plurality of plates and retrieving a given plate to a fixed location, b) a streaking mechanism wherein the streaking mechanism is directly attached to the plate carousel system, c) a sample dispensing system, and d) an optional wash station.

2. An apparatus of claim 1, wherein the plate carousel system comprises a plurality of removable multi-compartmental cassettes, a rotary platform adapted to house the removable multi-compartmental cassettes, and an integral plate picker mechanism capable of retrieving a plate from a multi-compartmental cassette and deliver the plate to a fixed location.

3. An apparatus of claim 2, wherein the streaking mechanism comprises a floating stylus, and a coupled multi-axis XYZ-Theta motion system.
